(19) United States
(12) Patent Application Publication
(10) Pub. No.: US 2026/0232857 A1
(43) Pub. Date: Aug. 13, 2026

(54) STERILIZATION DEVICE AND STERILIZATION METHOD USING SAME

(71) Applicant: HANSO INC., Daejeon (KR)

(72) Inventors: Sang Woong KIM, Daejeon (KR); Eun Ju LEE, Daejeon (KR); Chang Ick LEE, Cheongju-si (KR); Jun Seok SONG, Daejeon (KR)

(73) Assignee: HANSO INC., Daejeon (KR)

(21) Appl. No.: 19/475,201

(22) PCT Filed: Mar. 12, 2024
(86) PCT No.: PCT/KR2024/003169

§ 371 Date: Dec. 16, 2025

(30) Foreign Application Priority Data
Apr. 18, 2023 (KR) 10-2023-0050737

(51) Int. Cl. A61L 2/208; A61L 103/65

(52) U.S. Cl. CPC A61L 2/208; A61L 2103/65; A61L 2202/11; A61L 2202/122; A61L 2202/13; A61L 2202/15

(57) ABSTRACT

The present disclosure relates to a sterilization device that shortens sterilization time and improves efficiency by configuring a large-capacity chamber. The device includes a chamber with a sterilization space, a fluid channel separated from the wall, and a barrier with through-holes to allow fluid flow between the fluid channel and sterilization space. A sterilization module supplies sterilization gas to the fluid channel, and a catalyst module circulates air within the chamber. The fluid channel consists of a sterilization gas channel connected to the sterilization module and an internal circulation channel linked to the catalyst module, both separated for distinct functions.

### CROSS-REFERENCE TO RELATED APPLICATIONS

This application is a 35 U.S.C. § 371 national phase of PCT International Application No. PCT/KR 2024/003169, filed Mar. 12, 2024, which claims the benefit of priority under 35 U.S.C. § 119 to Republic of Korea Application No. 10-2023-0050737, filed Apr. 18, 2023, the content of which is incorporated herein by reference in its entirety.

### TECHNICAL FIELD

The present disclosure relates to a sterilization device capable of sterilizing and disinfecting a sterilization target with maximum sterilizing power, and a sterilization method using the same.

### BACKGROUND

Sterilization is the killing of microorganisms by applying physical and chemical stimuli. Sterilization methods include sterilization and disinfection. The sterilization is the killing of all microorganisms including both pathogenic and non-pathogenic to achieve a completely sterile state, while disinfection is the killing of pathogenic organisms to achieve a nearly sterile state.

The sterilization is used in various fields, including food, environment, construction, and transportation, and plays a particularly important role in the medical field, which deals with infectious microorganisms. Currently, the sterilization devices used in the medical field utilize various methods, including ultraviolet sterilization using wavelengths such as UV-C, steam sterilization using high-temperature and high-pressure steam, chemical sterilization using ethylene oxide gas, chloroquine acid, or the like, and plasma sterilization.

However, the UV sterilization has a limited sterilization range for objects with complex structures because UV rays may only travel in a straight line, and may be harmful to the human body depending on the exposure time and intensity. Furthermore, since the steam sterilization involves filling a sealed container with saturated steam, pressurizing the sealed container, and heating the sealed container to temperatures exceeding 100? C, the exposure of the sterilization target to high temperatures and moisture may cause product deformation. Furthermore, the chemical sterilization can be difficult to manage due to issues such as residual chemicals in devices, depending on the chemicals used, and may pose a risk of harmful side effects to patients or medical staffs.

To address these issues, a sterilization device using hydrogen peroxide has been proposed. The hydrogen peroxide may possess high sterilizing power and may be decomposed into water vapor and oxygen for discharge, making it safe and environmentally friendly.

However, the conventional hydrogen peroxide sterilization devices perform sterilization by creating a chamber containing objects to be sterilized into a vacuum state to allow hydrogen peroxide to diffuse therein. Therefore, to increase the efficiency of the hydrogen peroxide diffusion, the chamber size should be limited, resulting in limited capacity and a longer process time. Furthermore, creating the chamber into the vacuum state to diffuse the hydrogen peroxide requires considerable time, and the combination of the vacuum device and its components makes the maintenance of the device difficult and complicated.

An object of the present disclosure provides a sterilization device that is configured with a large-capacity chamber to contain a large number of sterilization targets for processing, shortens a sterilization process time through a structure that enables a sterilization fluid used for sterilization to be uniformly distributed throughout the inside of the chamber within a shorter period of time, and maximally increases sterilizing power through a structure that allows the sterilization fluid to be evenly distributed over all the sterilization target contained therein, and a sterilization method using the same.

In one general aspect, a sterilization device includes: a chamber including a sterilization space in which a sterilization target is contained, a fluid channel spaced apart inwardly from a wall surface by a predetermined distance and receiving a fluid from the outside, and a barrier separating the sterilization space, the barrier including multiple through-holes such that the fluid flows between the fluid channel and the sterilization space through the through-holes; a sterilization module communicating with the fluid channel so as to supply a sterilization gas to the fluid channel; and a catalyst module communicating with the fluid channel so as to circulate air inside the chamber, in which the fluid channel is configured such that the sterilization gas channel communicating with the sterilization module, and an internal circulation channel communicating with the catalyst module are separated from each other.

The sterilization module may be arranged above or below the sterilization space such that the sterilization gas channel communicates with the sterilization module at an upper or lower surface of the sterilization space.

The catalyst module may be arranged at one side of the sterilization space such that the internal circulation channel communicates with the catalyst module at a predetermined area of a side surface of the sterilization space.

The fluid channel may be formed on each surface by the barrier arranged along an inner side surface of the chamber, and the sterilization gas channel may be formed to have a larger area than the internal circulation channel.

The sterilization module may include: a first fan having an inlet formed therein to suck the fluid within the sterilization space; a first heater heating the fluid passing therethrough; and a vaporizer vaporizing the fluid passing therethrough.

The sterilization module may further include a hydrogen peroxide supply unit supplying a hydrogen peroxide solution to the vaporizer, and the hydrogen peroxide supply unit may operate only when the hydrogen peroxide is required.

The vaporizer may convert the hydrogen peroxide solution into vapor and a gaseous state and supply the vapor to the sterilization space through the sterilization gas channel.

The sterilization module may include an outlet that has one side formed by branching between the first heater and the vaporizer and the other side connected to the outside to discharge the passing fluid, and has an inside filled with a catalyst decomposing the fluid.

The catalyst module may include: a second fan sucking the fluid within the sterilization space; a second heater heating the fluid passing therethrough; and a catalyst cartridge decomposing the fluid passing therethrough; and a dehumidifier removing moisture from the fluid passing therethrough.

The catalyst cartridge may decompose hydrogen peroxide gas into the moisture and oxygen.

The catalyst module may include an inlet through which air within the sterilization space flows into the catalyst module and an outlet through which the fluid passing through the catalyst module is discharged into the sterilization space, and the inlet and the outlet may communicate with the internal circulation channel.

The internal circulation channel may be configured as a pair formed separately from each other on one side of an outer surface of the chamber, having a length in a height direction of the chamber with a predetermined area, and one of the pair may communicates with the inlet and the other may communicate with the outlet.

The barrier may include a plurality of protruding hangers arranged on a wall surface of the sterilization space to hang and load the sterilization target.

The barrier may include at least one through-hole arranged at a lower end of the protruding hanger.

In another general aspect, a sterilization method using the sterilization device includes: a preprocessing step in which a sterilization module operates to regulate air within the sterilization space to a preset temperature and humidity; a gas processing step in which, when the sterilization space is set to the preset temperature and humidity, a hydrogen peroxide supply unit supplies a hydrogen peroxide solution to a vaporizer contained in the sterilization module, the hydrogen peroxide solution is converted into vapor and a gaseous state by the vaporizer, and the hydrogen peroxide gas is supplied to the sterilization space through the sterilization gas channel; a sterilization step in which the sterilization target is sterilized by the hydrogen peroxide gas supplied to the sterilization space; and a ventilation step in which the catalyst module operates to cause air within the sterilization space to be sucked through the internal circulation channel, to pass through a catalyst cartridge, and to be decomposed into moisture and oxygen.

In the gas processing step, the air within the sterilization space may be re-circulated to the sterilization module for a preset period of time, and the hydrogen peroxide gas may be repeatedly supplied to the sterilization space.

In the ventilation step, the air passing through the catalyst cartridge may be circulated to the sterilization space through a dehumidifier contained in the sterilization module.

According to the sterilization device of the present disclosure configured as described above, and the sterilization method using the same, the large-capacity chamber may be configured to improve the efficiency of the sterilization process. And the gas circulation structure within the chamber allows the sterilizing agent solution to be more evenly diffused even in an appropriate environment rather than a vacuum, thereby improving the energy efficiency and ease of operation of the device, and shortening the time necessary for the sterilization process.

### BRIEF DESCRIPTION OF THE DRAWINGS

FIG. 1 is a perspective view of the entire sterilization device.

FIG. 2 is a perspective view of the sterilization device with a portion of the housing removed.

FIG. 3 is a cross-sectional view taken along line A-A′ of FIG. 2A.

FIG. 4 is a configuration diagram of a sterilization device module according to an embodiment of the present disclosure.

FIG. 5 is a cross-sectional view taken along line B-B′ of FIG. 2.

FIG. 6 is a detailed configuration diagram and fluid flow diagram of the sterilization module of the present disclosure.

FIG. 7 is a cross-sectional view taken along line C-C′ of FIG. 2.

FIG. 8 is a circuit diagram of a sterilization module of the present disclosure.

FIG. 9 is a detailed configuration diagram and fluid flow diagram of a catalyst module of the present disclosure.

FIG. 10 is a cross-sectional view taken along line D-D′ of FIG. 2.

FIG. 11 is a circuit diagram of the catalyst module of the present disclosure.

FIG. 12 is an internal view of a sterilization device into which a sterilization target is loaded using a loading means according to another embodiment of the present disclosure.

FIG. 13 is a flowchart of a sterilization method of the present disclosure.

### DETAILED DESCRIPTION

Hereinafter, a technical idea of the present disclosure will be described in more detail with reference to the accompanying drawings. Terms and words used in the present specification and claims are not to be construed as a general or dictionary meaning, but are to be construed as meaning and concepts meeting the technical ideas of the present disclosure based on a principle that the present inventors may appropriately define the concepts of terms in order to describe their inventions in best mode.

Therefore, configurations described in exemplary embodiments and the accompanying drawings of the present disclosure do not represent all of the technical spirits of the present disclosure, but are merely most preferable embodiments. Therefore, the present disclosure should be construed as including all the changes, and substitutions included in the spirit and scope of the present disclosure at the time of filing this application.

Hereinafter, a technical idea of the present disclosure will be described in more detail with reference to the accompanying drawings. The accompanying drawings are only examples shown in order to describe the technical idea of the present disclosure in more detail. Therefore, the technical idea of the present disclosure is not limited to shapes of the accompanying drawings.

The present disclosure relates to a sterilization device 1000, and relates to a device that sterilizes a sterilization target by loading a sterilization target into a chamber 100 and evenly diffusing a sterilizing agent within the chamber 100. In this case, the present disclosure is a device that configures the chamber 100 for loading the sterilization target to have a large capacity and configures a fluid channel 10 and a system such that the sterilizing agent may be more evenly diffused throughout the large-capacity chamber 100, thereby improving sterilizing power and improving the operation efficiency of the sterilization process.

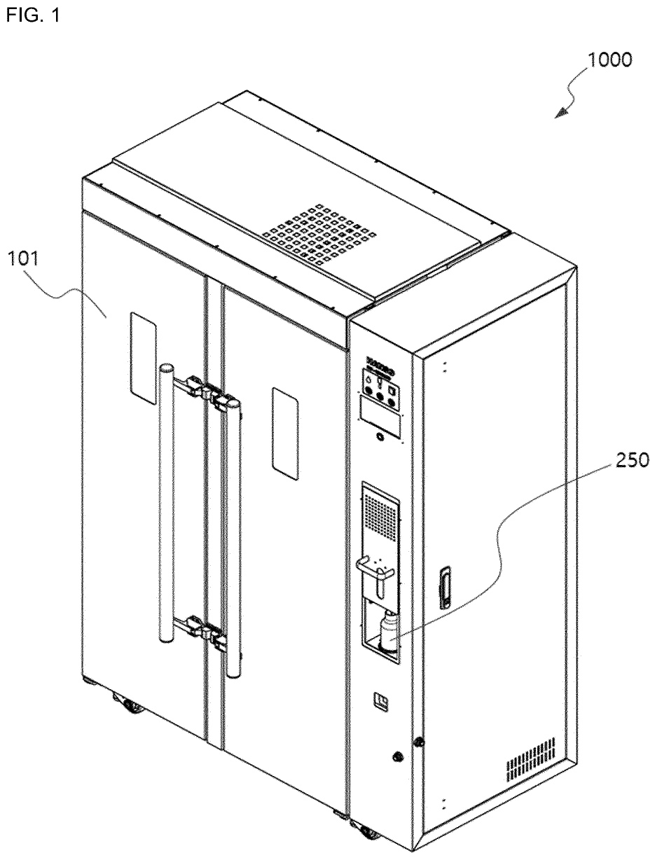

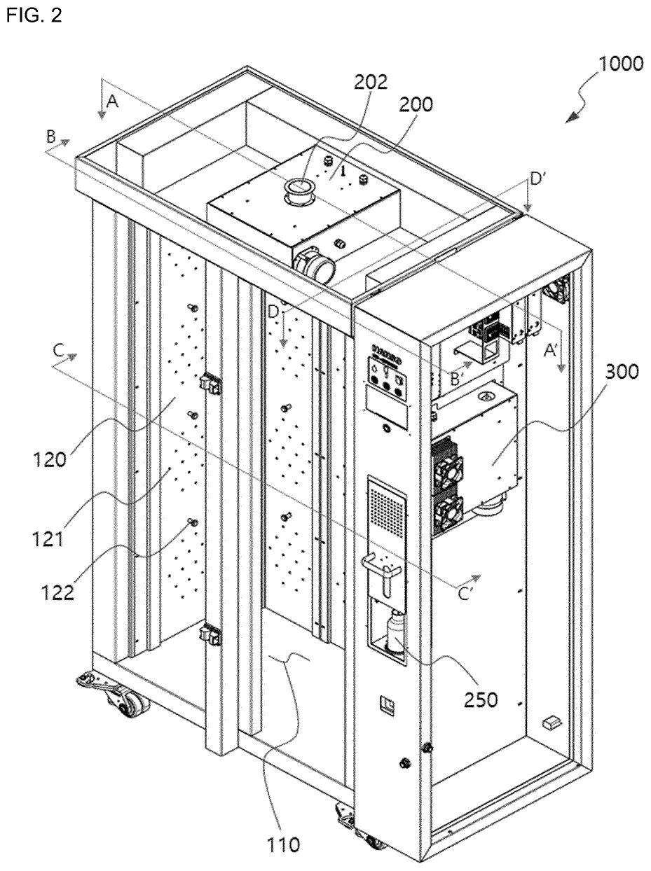

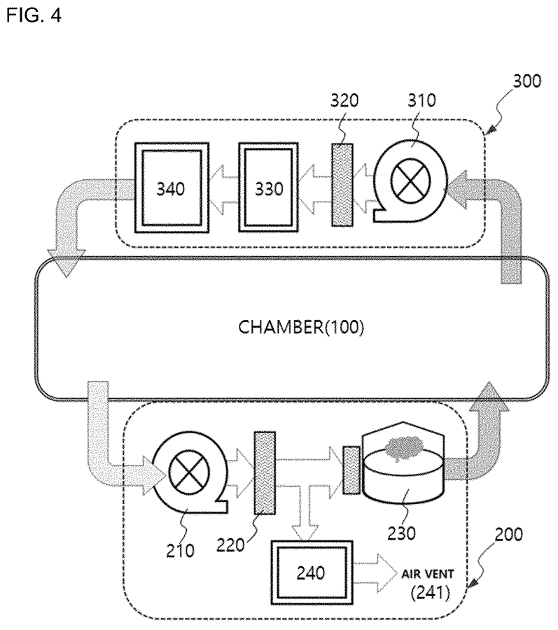

Referring to FIGS. 1 and 2, the sterilization device 1000 of the present disclosure includes a sterilization space 110 in which a sterilization target is contained, and a barrier 120 that is arranged inwardly from a wall surface by a predetermined distance to separate the sterilization space 110 from a fluid channel 10 that receives a fluid from the outside, in which the barrier 120 includes multiple through-holes 121, and includes the chamber 100 in which the fluid flows between the fluid channel 10 and the sterilization space 110 through the through-holes 121. As illustrated in FIG. 4, the sterilization device 1000 includes a sterilization module 200 that communicates with the fluid channel 10 and supplies sterilization gas to the fluid channel 10 and a catalyst module 300 that communicates with the fluid channel 10 and circulates air inside the chamber 100. In this case, the fluid channel 10 is configured such that a sterilization gas channel 11 that communicates with the sterilization module 200 and an internal circulation channel 12 that communicates with the catalyst module 300 are separated from each other.

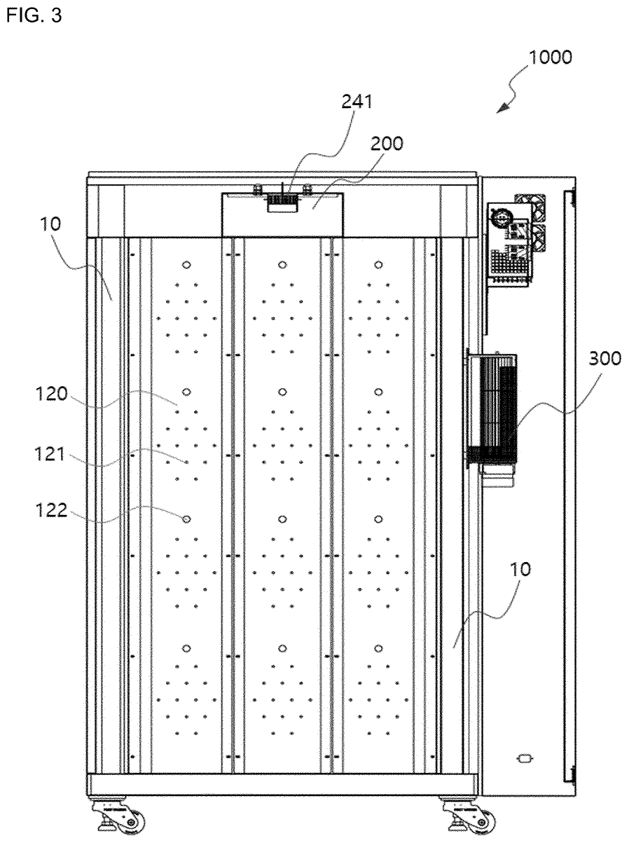

The sterilization device 1000 is configured to include the chamber 100 in which a sterilization process is performed. Referring to FIGS. 2 and 3, the chamber 100 includes the sterilization space 110 inside for containing the sterilization target, and is configured such that the fluid channel 10 that receives a fluid supply between the barrier 120 and the wall is separately formed through the barrier 120 arranged inwardly from the wall surface of the chamber 100 at a predetermined distance. In other words, the chamber 100 of the present disclosure is configured such that the barrier 120 is arranged along an inner circumferential surface of the chamber 100 at a predetermined distance from an inner wall surface of the chamber 100, the sterilization space 110 that contains the sterilization target is formed on the inside of the barrier 120, and the fluid channel 10 through which the fluid flows is formed between the barrier 120 and the inner wall surface of the chamber 100.

The chamber 100 may be formed without any external shape restrictions, such as a polygon or circle, as long as it may form a space inside. The sterilization device 1000 includes a door for loading a sterilization target into the sterilization space 110, and is configured such that the sterilization space 110 inside the chamber 100 is opened by opening the door. In an embodiment of the present disclosure, the sterilization device 1000 of the present disclosure may have a door formed on one of the side surfaces, and it is preferable that the door is structured to seal the inside of the sterilization space 110 when closed.

The barrier 120 is for separating the internal space of the chamber 100 into the fluid channel 10 and the sterilization space 110. Referring to FIGS. 2 and 3, the barrier 120 is preferably formed with a length capable of separating the space within the chamber 100. The barrier 120 may be selectively formed only in a portion of the chamber 100 where the fluid channel 10 is to be formed, or may be arranged over the entire inner circumferential surface of the chamber 100. In addition, the barrier 120 may be provided on upper, lower, and side surfaces of the chamber 100, respectively. In an embodiment of the present disclosure, the barrier 120 is arranged along the side surface of the chamber 100. Accordingly, the barrier 120 is formed with a length corresponding to a height of the chamber 100, and may be arranged to separate the space within the chamber 100 in a longitudinal direction.

The fluid channel 10 is a channel through which the fluid flows, and is intended to guide fluid supplied from the outside or the internal air of the sterilization space 110 in a certain direction. The fluid channel 10 is formed by the barrier 120 and is formed along the inner circumferential surface of the chamber 100. In this case, the fluid channel 10 may be formed to be separated from each other on each surface of the inner surface of the chamber 100. More specifically, the barrier 120 is arranged such that the fluid channels 10 separated from each other are each formed on each surface of the inner circumferential surface of the chamber 100. For example, when the outer surface of the chamber 100 of the present disclosure is square, the inner wall surface of the chamber 100 may include four side surfaces, and the barrier 120 may be arranged to be separated from each other so as to form an independent fluid channel 10 on each surface.

In this case, according to the present disclosure, the sterilization gas channel 11, which communicates with the sterilization module 200 to allow the sterilization gas formed by the sterilization module 200 to flow, and an internal circulation channel 12, which communicates with the catalyst module 300 to allow the air inside the chamber 100 to circulate, are each formed separately. That is, the barrier 120 is formed so that the sterilization gas channel 11 and the internal circulation channel 12 are not connected to each other but are separated, and are arranged such that different types of fluids may flow inside the fluid channel 10. Accordingly, according to an embodiment, the barrier 120 forms the fluid channel 10 that is separated from each other on each of the four sides. However, according to the present disclosure, since it is important for the sterilizing agent to diffuse into the sterilization space 110, it is preferable that the sterilization gas channel 11 be formed with a larger area than the internal circulation channel 12. In this case, the internal circulation channel 12 may be formed with a predetermined area on one side, and the sterilization gas channel 11 may be formed with a wide area on each side surface of the chamber 100 to supply the sterilization gas into the sterilization space 110. That is, the sterilization gas channel 11 and the internal circulation channel 12 may be simultaneously arranged on one surface. In this case, the sterilization gas channel 11 and the internal circulation channel 12 are configured as channels separated by a barrier 120 so that they may not flow with each other. In an embodiment of the present disclosure, the chamber 100 may be formed to have four side surfaces, a door that performs opening and closing may be formed on one side surface, and the barrier 120 may be arranged along the remaining three surfaces, thereby forming the fluid channel 10. In this case, at least one sterilization gas channel 11 may be arranged on each three surfaces, and the internal circulation channel 12 may be arranged on one of the surfaces together with the sterilization gas channel 11 while being separated therefrom.

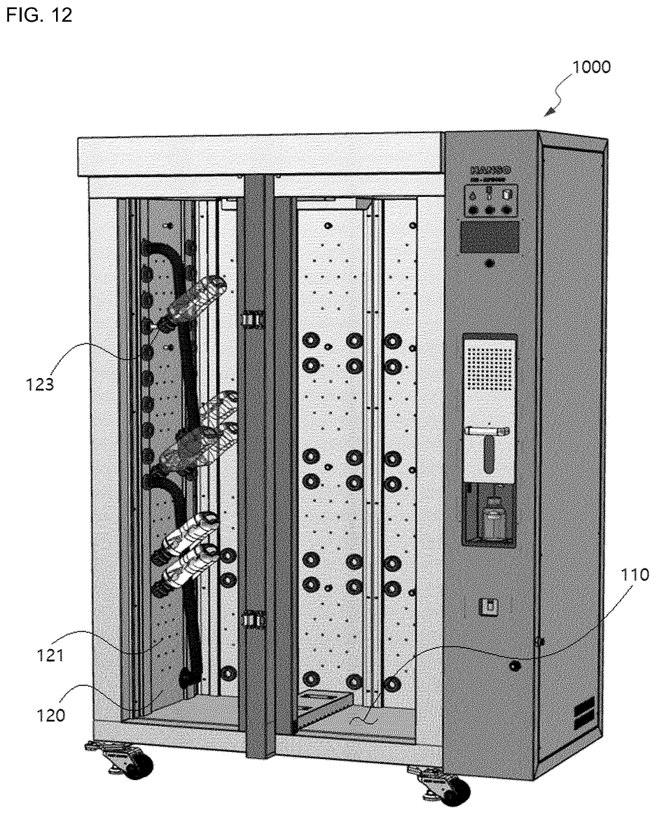

The inner space of the barrier 120 is the sterilization space 110, and the sterilization target is loaded into the sterilization space 110. In this case, it is preferable that the sterilization space 110 be provided with a loading means capable of loading the sterilization target. The loading means may be a shelf and a protruding hanger 122 for hanging loading, and may be configured without limitation as long as it has a structure into which the sterilization target may be loaded. It is preferable that the loading means is formed in a shape that is easy to load according to the shape of the sterilization target. As an embodiment of the present disclosure, referring to FIGS. 2 and 3, the loading means of the present disclosure may be configured with the protruding hanger 122, and the protruding hangers 122 may be configured in multiple numbers and may be freely arranged on the inner wall facing the sterilization space 110, which is the inner circumferential surface of the barrier 120. In this case, the number and spacing of the protruding hangers 122 may be freely configured and arranged according to the shape of the sterilization target or the needs of the sterilization device 1000. In addition, when the space inside the sterilization space 110 is wide, the protruding hanger 122 may have a structure in which one or more protruding hangers in the shape of a hanger, each having a plurality of branches formed on a long rod, are arranged. In addition, in an embodiment of the present disclosure, the sterilization target may be an article that may be used by sterilizing a surface of an article, and in particular, may be respiratory protection equipment such as a military gas mask, a firefighting gas mask, and a special gas mask for tanks and aircraft, which are personal protective equipment. Accordingly, in another embodiment, the loading means is formed as a fixed hanger in the shape of a tube, so that the sterilization target of the respiratory protection equipment or the sterilization target including a container or hose structure may be loaded into the chamber by inserting into or hanging on the fixed hanger. Referring to FIG. 12, the loading means may be configured to attach and detach various shaped tools to and from the barrier to sterilize personal equipment such as water bottles, helmets, and vests. For example, the loading means may be a tube-shaped hanger 123, and a water bottle may be loaded by being inserted into the tube, and sterilized by supplying the sterilization gas to the inside. In addition, the gas masks used by tank soldiers and the front mask gas masks used by aircraft pilots may be formed to sterilize the inside of the tube-shaped supply pipe that supplies air passing through a positive pressure device by connecting the tube-shaped supply pipe to a dedicated connection hole.

In addition, the barrier 120 includes multiple through-holes 121. The through-hole 121 is for allowing the fluid to flow between the fluid channel 10 and the sterilization space 110, and it is preferable that the through-hole 121 is formed in multiple numbers. Referring to FIGS. 2 and 3, even when the fluid channels 10 of the present disclosure are formed to be separated from each other by a barrier 120, the sterilization space 110 and the inside of the fluid channel 10 may be connected through the through-hole 121. The size, number, and arrangement of the through-holes 121 may be freely set, but in order to evenly diffuse the sterilizing agent flowing along the fluid channel 10 formed by the barrier 120 over the entire area of the sterilization space 110, it is preferable that the through-holes 121 be arranged so as to be evenly diffused over the entire area of the barrier 120. In particular, when each protruding hanger 122 is arranged on the barrier 120, since the sterilization target is hung and loaded onto the protruding hanger 122, it is preferable that the through-holes 121 are arranged in greater numbers near the protruding hanger 122 than in other locations. In particular, it is preferable that at least one or more through-holes 121 are arranged on the lower side of the protruding hanger 122.

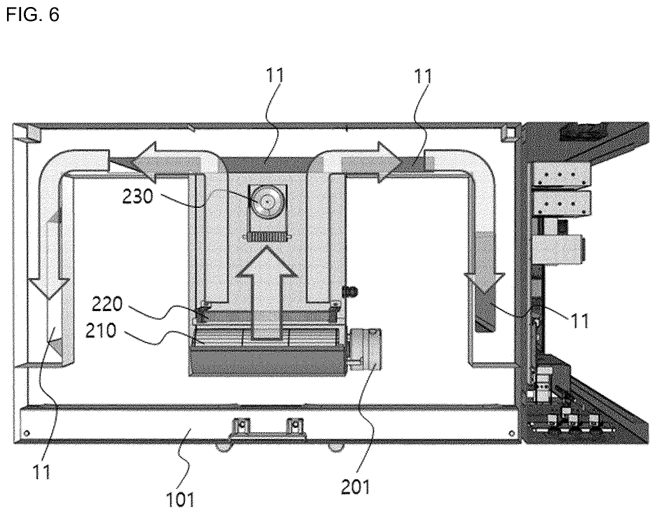

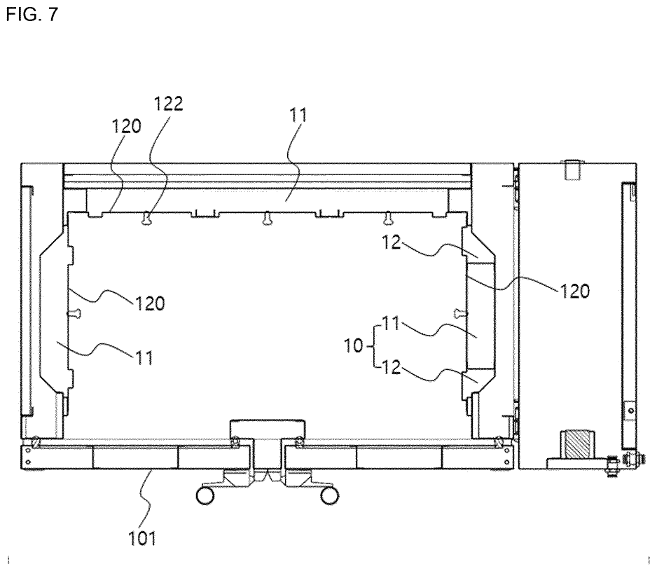

The sterilization module 200 is a device that supplies the sterilization gas to the sterilization space 110 through the fluid channel 10. That is, the sterilization module 200 generates the sterilization gas and supplies the sterilization gas to the sterilization space 110, and it is preferable that the sterilization module 200 be formed to communicate with at least one separate fluid channel 10. Referring to FIGS. 3 and 4, the sterilization module 200 communicates with the sterilization gas channel 11, and operates by supplying air into the sterilization space 110 through the sterilization gas channel 11 or receiving air from the inside. In this case, the sterilization module 200 may be arranged on the upper or lower side of the sterilization space 110, and the fluid channel 10 is formed to communicate with the sterilization module 200 on the upper or lower surface of the sterilization space 110 depending on the arrangement position of the sterilization module 200. As an embodiment of the present disclosure, referring to FIGS. 6 and 7, the sterilization module 200 may be arranged on the upper side of the sterilization space 110, and the sterilization module 200 may be formed with a separate inlet 201 for sucking the air of the sterilization space 110. The air inside the sterilization space 110 sucked through the inlet 201 moves to the fluid channel 10 after passing through the sterilization module 200. Accordingly, the sterilization gas channel 11 among the fluid channels 10 formed by the barrier 120 of the chamber 100 is formed to communicate with the sterilization module 200 on the upper surface of the sterilization space 110. In more detail, the sterilization space 110 forms the plurality of fluid channels 10 in the side portion by the barrier 120, and the fluid channels 10 are formed to have channels in the height direction of the sterilization space 110. Accordingly, among the fluid channels 10, the sterilization gas channels 11 are formed with their upper surfaces open, and the sterilization module 200 is arranged on the upper surface, so the open upper surface of the sterilization gas channel 11 and the sterilization module 200 communicate with each other, and the sterilization module 200 and the fluid channel 10 may be connected to each other. Accordingly, according to the present disclosure, the gas that has passed through the sterilization module 200 flows from the upper side of the fluid channel 10 through the sterilization gas channel 11, flows downward along the inner surface of the chamber 100, and diffuses into the sterilization space 110 through each through-hole 121 of the barrier 120, thereby causing the gas to flow.

Referring to FIGS. 4, 6, and 8, the sterilization module 200 includes an inlet 201, a first fan 210 (blower) configured to suction at least the fluid inside the sterilization space 110, a first heater 220 (PTC) for heating the fluid, and a vaporizer 230 for vaporizing the fluid. Accordingly, the sterilization module 200 may suck the air of the sterilization space 110 through the inlet 201 due to the operation of the first fan 210, heat the fluid that has passed through the first fan 210 by the first heater 220, and vaporize the heated fluid by the vaporizer 230, thereby forming a gas. By sending this out to the sterilization space 110 through the sterilization gas channel 11, the inside of the sterilization space 110 is formed into an environment of a certain standard, thereby preheating and preparing the sterilization device 1000. That is, the sterilization module 200 may heat the inside of the sterilization space 110 to a set temperature while circulating the air inside the sterilization space 110.

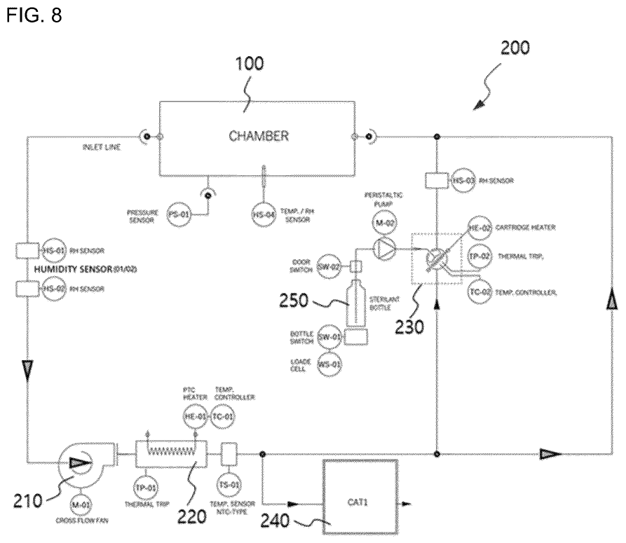

In addition, the sterilization module 200 of the present disclosure may further include a hydrogen peroxide supply unit 250 that supplies a hydrogen peroxide solution to the vaporizer 230. As illustrated in FIG. 2, the hydrogen peroxide supply unit 250 may be arranged outside the chamber 100 and configured to operate only when necessary. Referring to FIG. 8, the hydrogen peroxide supply unit 250 may contain the hydrogen peroxide solution used as the sterilizing agent, may store a predetermined hydrogen peroxide solution in a container, may include a pump structure, and may be configured without limitation as long as it may deliver the hydrogen peroxide solution to the vaporizer 230 according to a signal when necessary. When the hydrogen peroxide solution is required, the sterilization space 110 may be configured as a high-temperature environment, and accordingly, the hydrogen peroxide supply unit 250 may supply the hydrogen peroxide solution to the vaporizer 230. Accordingly, the hydrogen peroxide supply unit 250 may be configured to provide the hydrogen peroxide solution to the vaporizer 230. The vaporizer 230 vaporizes the hydrogen peroxide solution supplied from the hydrogen peroxide supply unit 250, converting the hydrogen peroxide solution into vapor and gas, and supplies the hydrogen peroxide solution to the sterilization space 110 through the sterilization gas channel 11. The hydrogen peroxide gas converted into a vapor and gas state is supplied to the sterilization space 110.

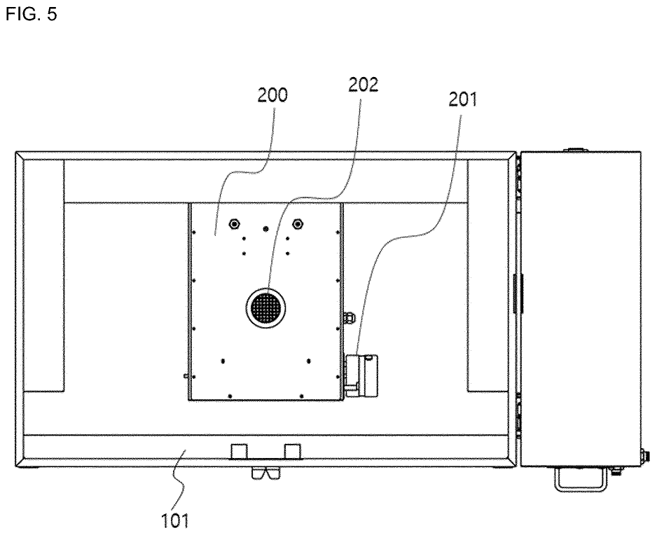

Referring to FIGS. 2, 4, and 5, the sterilization module 200 of the present disclosure may further include an outlet 220 which branches off from between the first heater 220 and the vaporizer 230 on one side and is connected to the outside of the device on the other side to discharge the passing fluid to the outside. In this case, the outlet 220 may be filled with a catalyst inside, and the catalyst may be a catalyst 240 (CAT) that decomposes the passing fluid. The catalyst unit 240 is located inside the outlet 202, and is arranged between the first heater 220 and the vaporizer 230, and the air inside the sterilization space 110 is sucked through the inlet 201, passes through the first heater 220, and then flows in along the branch portion to pass through the catalyst unit 240, thereby decomposing the air into water vapor and oxygen. The outlet 202 has one side connected between the first heater and the vaporizer, and the other side connected to the outside of the chamber 100. The decomposed water vapor and oxygen pass through the catalyst unit 240 and are discharged to the outside of the device through the outlet 202. The outlet 202 is formed separately from the inlet 201 or the part connected to the sterilization gas channel 11. In this case, when the first fan 210 (blower) operates, a lower pressure than the inside of the chamber 100 may be generated at the front end (intake) of the first fan 210, and a higher pressure than the inside of the chamber 100 may be generated at the rear end (outlet) of the first fan 210. In this case, the pressure difference generated by the operation of the first fan 210 may be used to maintain the pressure inside the chamber 100 lower than the pressure outside the chamber 100. The outlet 202 connected to the outside of the chamber 100 is installed at the rear end of the first fan 210, so that while the first fan 210 is operating, the pressure inside the chamber 100 is maintained lower than the pressure outside the chamber 100, thereby preventing the sterilizing gas inside the sterilization space 110 from leaking out of the chamber 100 due to the pressure difference. In addition, the pressure difference in the chamber due to the operation of the first fan 210 may prevent the sterilization gas from leaking out through the gap of the chamber 100. In addition, when the pressure inside the chamber 100 becomes higher than the external pressure, the sterilizing gas and air inside the chamber 100 are decomposed into moisture and oxygen while passing through the catalyst unit 240 through the outlet 202 installed at the rear end of the first fan 210 and discharged to the outside, thereby continuously maintaining the pressure inside the chamber 100 lower than the external pressure.

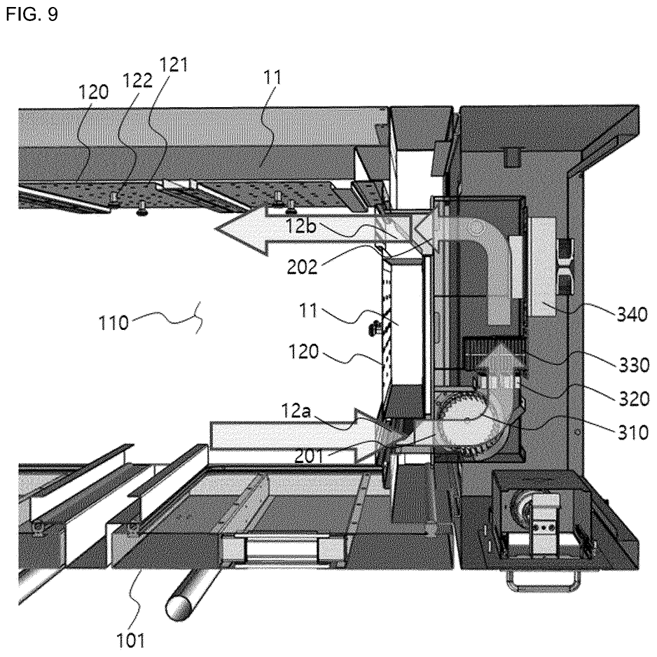

The catalyst module 300 is a device that circulates the internal air of the chamber 100 through the fluid channel 10. The catalyst module 300 sucks the air inside the chamber 100, converts the air, and supplies the air to the chamber 100 as reusable air. The fluid passing through the catalyst module 300 flows through the internal circulation channel 12. Referring to FIGS. 2 and 4, the catalyst module 300 communicates with at least one internal circulation channel 12, which is the separate fluid channel 10, and operates by sending air into the sterilization space 110 or receiving air from the inside through the internal circulation channel 12. In this case, the catalyst module 300 may be arranged on one side surface of the sterilization space 110 along the barrier 120, and the internal circulation channel 12 is formed to communicate with the catalyst module 300 in a predetermined area on the side of the sterilization space 110, depending on the arrangement position of the catalyst module 300. As an embodiment of the present disclosure, referring to FIG. 9, the catalyst module 300 may be arranged on one side of the sterilization space 110, and the catalyst module 300 may include an inlet 301 and an outlet 302 through which air moves to and from the sterilization space 110 via the internal circulation channel 12. More specifically, the catalyst module 300 may be arranged on the outer surface of the sterilization space 110, and the inlet 301 and outlet 302 may be formed in a portion of the catalyst module 300 attached to the sterilization space 110. The inlet 301 and outlet 302 are formed to communicate with each of the internal circulation channels 12.

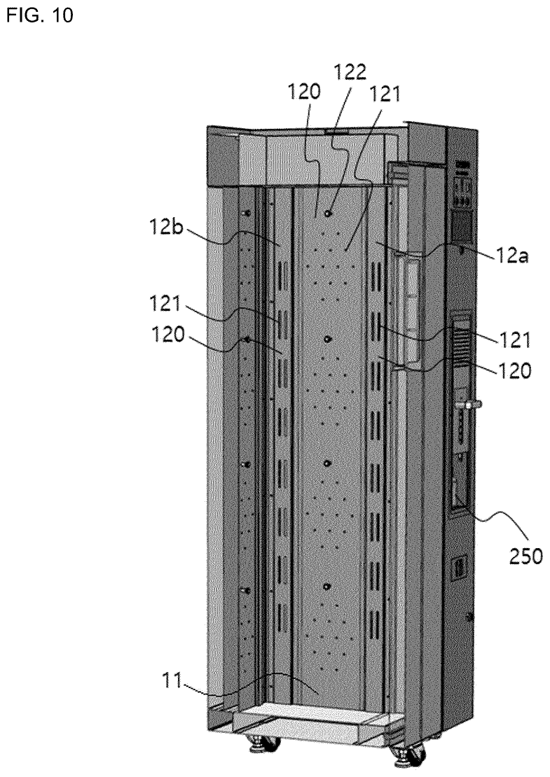

Referring to FIGS. 9 and 10, the internal circulation channel 12 of the present disclosure is formed on one of the side surfaces of the chamber 100, and has a channel in the height direction of the chamber 100 with a predetermined cross-sectional area. Furthermore, the inlet 301 and outlet 302 are formed in a portion along the length of the internal circulation channel 12 so as to be connected to the catalyst module 300. Accordingly, the air inside the sterilization space 110 flows into the internal circulation channel 12 from the through-hole 121 of the barrier 120 and is sucked into the catalyst module 300 through the inlet 301. Then, the air passing through the catalyst module 300 flows into the internal circulation channel 12 through the outlet 302 and is introduced into the sterilization space 110 through the through-hole 121 of the barrier 120. In this case, as an embodiment of the present disclosure, as illustrated in FIG. 10, the internal circulation channel 12 may be formed as a pair of fluid channels 10 arranged in parallel with a predetermined area on both sides of a the sterilization gas channel 11 formed on one side of the chamber 100. A pair of the internal circulation channels 12 is formed to be separated from each other by the fluid channel 10, and is also separated from the sterilization gas channel 11. In addition, one of a pair of internal circulation channels 12 may be connected to the inlet 301, and the other may be connected to the outlet 302. That is, among the pair of internal circulation channels 12, the first internal circulation channel 12a communicating with the inlet 301 may be used as a channel for introducing air inside the sterilization space 110 through the inlet 301 into the catalyst module 300. In addition, the other second internal circulation channel 12b connected to the outlet 302 may be used as a channel for discharging air that has passed through the catalyst module 300 through the outlet into the sterilization space 110. Accordingly, according to the present disclosure, the air inside the sterilization space 110 flows into the first internal circulation channel 12a through the through-hole 121 formed along the barrier 120, and is then sucked into the catalyst module 300 through the inlet 301. Then, the air filtered while passing through the catalyst module 300 flows into the second internal circulation channel 12b connected to the outlet 302 through the outlet 302, and is diffused into the sterilization space 110 through the through-hole 121 formed along the barrier 120 of the second internal circulation channel 12b, thereby circulating the gas.

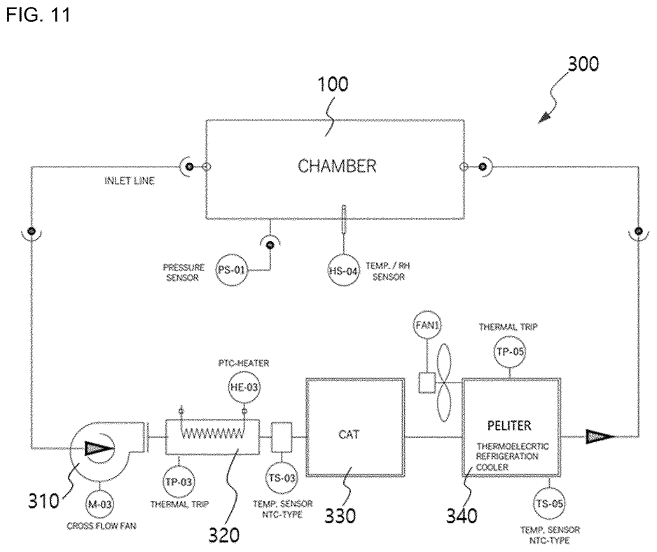

Referring to FIGS. 4, 9, and 11, the catalyst module 300 of the present invention includes at least a second fan 310 (blower) for sucking the fluid inside the sterilization space 110, a second heater 320 (PTC) for heating the passing fluid, a catalyst cartridge 330 (CAT) for decomposing the fluid, and a dehumidifier 240 (Peltier) for removing moisture from the passing fluid. Accordingly, the catalyst module 300 sucks air from the sterilization space 110 through the inlet due to the operation of the second fan 310, the second heater 320 heats the fluid passing through the second fan 310. The heated fluid passes through the catalyst cartridge 330 and decomposed into specific components, and the dehumidifier 240 removes moisture from the decomposed fluid to filter the air by removing certain components. In this case, the catalyst cartridge 330 is for decomposing the sterilization gas remaining inside the sterilization space 110, and decomposes the hydrogen peroxide gas into moisture and oxygen. In addition, it is preferable that the dehumidifier 240 is heated to a predetermined reference temperature for moisture removal, and thus, the air passing through the dehumidifier 240 may be circulated into the sterilization space 110 in a reheated state.

Hereinafter, a sterilization method using the sterilization device 1000 of the present disclosure having the above-described characteristics will be described.

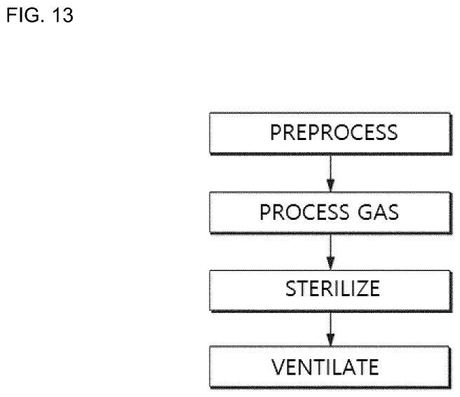

Referring to FIG. 13, the sterilization method of the present disclosure includes a preprocessing step in which the sterilization module 200 and the catalyst module 300 operate to regulate air within the sterilization space to a preset temperature and humidity; a gas processing step in which, when the sterilization space 110 is set to the preset temperature and humidity, the hydrogen peroxide supply unit 250 supplies a hydrogen peroxide solution to the vaporizer 230 contained in the sterilization module 200, the hydrogen peroxide solution is converted into vapor and a gaseous state by the vaporizer 230, and the hydrogen peroxide gas is supplied to the sterilization space 110 through the sterilization gas channel 11; a sterilization step in which the sterilization target is sterilized by the hydrogen peroxide gas supplied to the sterilization space 110; and a ventilation step in which the catalyst module 300 operates to cause air within the sterilization space 110 to be sucked through the internal circulation channel 12, to pass through a catalyst cartridge 330, and to be decomposed into moisture and oxygen.

In the sterilization method of the present disclosure, the sterilization device 1000 is a device capable of removing biological contamination of the sterilization target by supplying hydrogen peroxide gas, which is a sterilizing agent, into the device and uniformly diffusing and circulating the hydrogen peroxide gas within the device. First, when the sterilization device 1000 is powered on and the system is turned on, a system preheating and preparing step, in which the sterilization module 200 and the catalyst module 300 operate to create an environment inside the sterilization space 110 at a preset temperature and humidity, is performed.

Through the system preheating and preparation step, the temperature and humidity within the sterilization space 110 are maintained at a constant level to prepare for the subsequent sterilization process. In this case, the internal temperature of the sterilization space 110 may be between 40 and 50° C. and the humidity may be 30% or less.

Furthermore, the sterilization method of the present disclosure may perform a self-diagnosis step prior to the preprocessing step. This self-diagnosis step is a step for confirming whether the sterilization device 1000 is abnormal and may be optionally performed by the operator. By performing this self-diagnosis step prior to the preprocessing step, it is possible to confirm whether the sterilization device 1000 is abnormal.

In the sterilization method of the present disclosure, when the system preheating and preparation step is completed, the preprocessing step is performed in which the sterilization target is loaded into the sterilization space 110 inside the chamber 100, and the sterilization module 200 operates to preheat both the sterilization space 110 and the sterilization target, thereby creating the environment having the preset temperature and humidity. The preprocessing step is a step of creating (conditioning) an optimal sterilization environment of the sterilization space 110 where the sterilization work is performed and the sterilization target. First, the step of creating the temperature set in the preprocessing step is performed by operating the sterilization module 200, so the air inside the sterilization space 110 is introduced into the sterilization module 200 through the inlet 201 on the upper side of the sterilization space 110. Air passing through each of the first heaters 220 and vaporizers 230 is heated and then introduced into the sterilization gas channel 11 that communicates with the sterilization device 1000. Accordingly, the heated air flows downward along the sterilization gas channel 11 and inputs the sterilization space 110 through the through-hole 121 formed in the barrier 120, thereby circulating the air inside the sterilization space 110. In addition, the preprocessing step may operate the catalyst module 300 to simultaneously create the preset humidity inside the sterilization space 110. Accordingly, the air of the sterilization space 110 is sucked into the catalyst module 300, and after sequentially passing through the second heater 320, the catalyst cartridge 330, and the dehumidifier 240, the dehumidified air may be circulated inside the sterilization space 110. In this case, the air with high humidity inside the sterilization space 110 is introduced into the fluid channel 10 through the through-hole 121 of the first internal circulation channel 12, and then introduced into the catalyst module 300 through the inlet 301 of the catalyst module 300. The introduced air passes through the second heater 320, the catalyst cartridge 330, and the dehumidifier 240, moves to the outlet 302 of the catalyst module 300, flows into the second internal circulation channel 12 communicating with the outlet 302, and then circulates the filtered air into the sterilization space 110 through the through-hole 121 of the second internal circulation channel 12. In this case, the second heater 320 does not operate. Therefore, the air dehumidified by passing through the dehumidifier 240 by the catalyst module 300 may be introduced into the sterilization space 110. By removing moisture in the sterilization space 110 by the dehumidifier 240, the humidity inside the sterilization space 110 may be controlled. That is, the preprocessing step is a step of circulating the air of the sterilization space 110 using the sterilization module 200 and the catalyst module 300 to create the sterilization chamber and the sterilization target at the set temperature and humidity. In this case, the preprocessing step may be repeatedly performed so that the sterilization space 110 has the temperature and humidity within a preset range. Accordingly, the sterilization space 110 is a step that may create an internal environment for the subsequent step and maintain and control the created humidity and temperature. When the gas processing step is performed without the preprocessing step, the sterilization target is not preheated and the temperature difference between the sterilization target and the inside of the sterilization space 110 is 10° or more, the sterilization gas supplied in the gas processing step may condense on the sterilization target, which reduces the sterilization efficiency, and the condensation of the sterilization gas may cause damage to the sterilization target. In addition, when the humidity is high, the sterilizing gas supplied in the gas processing step easily condenses with moisture inside the sterilization space 110, thereby reducing the sterilization efficiency. In order to prevent such condensation of the sterilizing gas and moisture, the operating sterilization device 1000 is characterized by a step of stabilizing the device by heating and preheating. In addition, the preprocessing step is stopped when the humidity and temperature inside the sterilization space 110 are set to a preset range, and the next step is performed.

In the sterilization method of the present disclosure, when the humidity and temperature within the sterilization space 110 are formed within a preset range, the preprocessing step is stopped, and the gas processing step of generating the sterilization gas and supplying (gassing) the sterilization gas to the sterilization space 110 is performed. The gas processing step is performed by the operation of the sterilization module 200. In more detail, when the sterilization space 110 is formed into a certain environment, the sterilization device 1000 operates the hydrogen peroxide supply unit 250 through the gas processing step to supply the hydrogen peroxide solution to the vaporizer 230. In this case, the hydrogen peroxide supply unit 250 may operate so that when the temperature of the second heater 320 or the vaporizer 230 of the sterilizer is heated to a set temperature, the solution pump operates to supply the hydrogen peroxide solution to the vaporizer 230. The vaporizer 230, which is heated to a certain temperature or higher, converts the supplied hydrogen peroxide solution into the vapor and gaseous state while passing through the vaporizer 230. In addition, the hydrogen peroxide gas passing through the vaporizer 230 is introduced into the fluid channel 10 through the sterilization gas channel 11 that communicates with the upper side of the sterilization module 200 and the sterilization space 110, and is diffused into the sterilization space 110. In this case, the gas processing step may be set to supply the sterilization gas to the sterilization space 110 by repeating the step during the set process time or process conditions. As the above gas processing step is repeatedly performed, the gas inside the sterilization space 110 is re-circulated to the sterilization module 200 through the vaporizer 230, thereby allowing the sterilization gas to be repeatedly supplied.

Thereafter, the sterilization step is performed in which the sterilization target is sterilized and disinfected by the sterilization gas supplied to the sterilization space 110. In the sterilization step, the hydrogen peroxide gas supplied into the sterilization space 110 by the gas treatment step flows into the sterilization gas channel 11 through the upper open portion of the sterilization gas channel 11, and is diffused into the sterilization space 110 through the through-hole 121 of the barrier 120 while flowing downward along the outer wall of the chamber 100 and the inside of the barrier 120. In this case, the hydrogen peroxide gas supplied to the sterilization space 110 is evenly distributed and diffused through the through-hole 121, thereby reaching the loaded sterilization target and sterilizing the contaminated sterilization target. In this case, in an embodiment of the present disclosure, the plurality of protruding hangers 122 for hanging and loading the sterilization target are arranged on the barrier 120, and the through-holes 121 are arranged together at the lower side of the protruding hangers 122. That is, the sterilization device 1000 of the present disclosure has a structure in which the sterilization target loaded by being hung on the protruding hanger 122 is positioned in a region where the through-holes 121 are gathered, and the hydrogen peroxide gas discharged through the through-holes 121 may directly contact the sterilization target. Since the hydrogen peroxide gas sufficiently supplied inside the sterilization space 110 is evenly diffused and distributed, even if the shape of the sterilization target is complex, the hydrogen peroxide gas may penetrate and sterilize every part of the sterilization target, thereby increasing the sterilizing power and reducing the time required for the sterilization process.

In addition, in the sterilization method of the present disclosure, after the sterilization step is performed, the catalyst module 300 operates to perform a ventilation step for ventilating the air inside the sterilization space 110. The ventilation step sucks air inside the sterilization space 110 into the catalyst module 300, passes the air through the catalyst cartridge 330, to decompose the air inside the sterilization space 110 into specific components, and re-circulates the air inside the sterilization space 110 into the sterilization space 110 as needed, thereby enabling the ventilation of the sterilization space 110. To explain in more detail, the ventilation step is such that the catalyst module 300 operates to suck the sterilizing gas inside the sterilization space 110 into the catalyst module 300, and after sequentially passing through the second heater 320, the catalyst cartridge 330, and the dehumidifier 240, the filtered air may be circulated into the sterilization space 110. In this case, the sterilization gas inside the sterilization space 110 is introduced into the fluid channel 10 through the through-hole 121 of the first internal circulation channel 12, and then introduced into the catalyst module 300 through the inlet 301 of the catalyst module 300. The introduced air passes through the second heater 320, the catalyst cartridge 330, and the dehumidifier 240, moves to the outlet 302 of the catalyst module 300, flows into the second internal circulation channel 12 communicating with the outlet 302, and then circulates the filtered air into the sterilization space 110 through the through-hole 121 of the second internal circulation channel 12. The catalyst cartridge 330 decomposes the hydrogen peroxide gas into moisture and oxygen, and the filtered air may be reheated through the dehumidifier 240 and introduced into the sterilization space 110. In addition, the humidity inside the sterilization space 110 may be controlled by removing the gas moisture in the sterilization space 110 by the dehumidifier 240.

Hereinabove, although the present disclosure has been described by specific matters such as detailed components and the accompanying drawings, they have been provided only for assisting in the entire understanding of the present disclosure. Therefore, the present disclosure is not limited to the embodiments. Various modifications and changes may be made by those skilled in the art to which the present disclosure pertains from this description.

Therefore, the spirit of the present disclosure should not be limited to these embodiments, but the claims and all of modifications equal or equivalent to the claims are intended to fall within the scope and spirit of the present disclosure.

1. A sterilization device, comprising:
a chamber including a sterilization space in which a sterilization target is contained, a fluid channel spaced apart inwardly from a wall surface by a predetermined distance and receiving a fluid from the outside, and a barrier separating the sterilization space, the barrier including multiple through-holes such that the fluid flows between the fluid channel and the sterilization space through the through-holes;
a sterilization module communicating with the fluid channel so as to supply a sterilization gas to the fluid channel; and
a catalyst module communicating with the fluid channel so as to circulate air inside the chamber,
wherein the fluid channel is configured such that the sterilization gas channel communicating with the sterilization module, and an internal circulation channel communicating with the catalyst module are separated from each other.

2. The sterilization device of claim 1, wherein the sterilization module is arranged above or below the sterilization space such that the sterilization gas channel communicates with the sterilization module at an upper or lower surface of the sterilization space.

3. The sterilization device of claim 1, wherein the catalyst module is arranged at one side of the sterilization space such that the internal circulation channel communicates with the catalyst module at a predetermined area of a side surface of the sterilization space.

4. The sterilization device of claim 2, wherein the fluid channel is formed on each surface by the barrier arranged along an inner side surface of the chamber, and the sterilization gas channel is formed to have a larger area than the internal circulation channel.

5. The sterilization device of claim 1, wherein the sterilization module includes:
a first fan having an inlet formed therein to suck the fluid within the sterilization space;
a first heater heating the fluid passing therethrough; and
a vaporizer vaporizing the fluid passing therethrough.

6. The sterilization device of claim 5, wherein the sterilization module further includes a hydrogen peroxide supply unit supplying a hydrogen peroxide solution to the vaporizer, and
the hydrogen peroxide supply unit operates only when the hydrogen peroxide is required.

7. The sterilization device of claim 6, wherein the vaporizer converts the hydrogen peroxide solution into vapor and a gaseous state and supplies the vapor to the sterilization space through the sterilization gas channel.

8. The sterilization device of claim 5, wherein the sterilization module includes an outlet that has one side formed by branching between the first heater and the vaporizer and the other side connected to the outside to discharge the passing fluid, and has an inside filled with a catalyst decomposing the fluid.

9. The sterilization device of claim 1, wherein the catalyst module includes:
a second fan sucking the fluid within the sterilization space;
a second heater heating the fluid passing therethrough;
a catalyst cartridge decomposing the fluid passing therethrough; and
a dehumidifier removing moisture from the fluid passing therethrough.

10. The sterilization device of claim 9, wherein the catalyst cartridge decomposes hydrogen peroxide gas into the moisture and oxygen.

11. The sterilization device of claim 9, wherein the catalyst module includes an inlet through which air within the sterilization space flows into the catalyst module, and an outlet through which the fluid passing through the catalyst module is discharged into the sterilization space, and
the inlet and the outlet communicate with the internal circulation channel.

12. The sterilization device of claim 11, wherein the internal circulation channel is configured as a pair formed separately from each other on one side of an outer surface of the chamber, having a length in a height direction of the chamber with a predetermined area, and one of the pair communicates with the inlet and the other communicates with the outlet.

13. The sterilization device of claim 1, wherein the barrier includes a plurality of protruding hangers arranged on a wall surface of the sterilization space to hang and load the sterilization target.

14. The sterilization device of claim 13, wherein the barrier includes at least one through-hole arranged at a lower end of the protruding hanger.

15. A sterilization method using the sterilization device of claim 1, comprising:
a preprocessing step in which a sterilization module operates to regulate air within the sterilization space to a preset temperature and humidity;
a gas processing step in which, when the sterilization space is set to the preset temperature and humidity, a hydrogen peroxide supply unit supplies a hydrogen peroxide solution to a vaporizer contained in the sterilization module, the hydrogen peroxide solution is converted into vapor and a gaseous state by the vaporizer, and the hydrogen peroxide gas is supplied to the sterilization space through the sterilization gas channel;
a sterilization step in which the sterilization target is sterilized by the hydrogen peroxide gas supplied to the sterilization space; and
a ventilation step in which the catalyst module operates to cause air within the sterilization space to be sucked through the internal circulation channel, to pass through a catalyst cartridge, and to be decomposed into moisture and oxygen.

16. The sterilization method of claim 15, wherein, in the gas processing step, the air within the sterilization space is re-circulated to the sterilization module for a preset period of time, and the hydrogen peroxide gas is repeatedly supplied to the sterilization space.

17. The sterilization method of claim 15, wherein, in the ventilation step, the air passing through the catalyst cartridge is circulated to the sterilization space through a dehumidifier contained in the sterilization module.

18. The sterilization device of claim 3, wherein the fluid channel is formed on each surface by the barrier arranged along an inner side surface of the chamber, and the sterilization gas channel is formed to have a larger area than the internal circulation channel.
